(19) United States
(12) Patent Application Publication
(10) Pub. No.: US 2025/0163367 A1
(43) Pub. Date: May 22, 2025

(54) THREE-DIMENSIONAL CELL CULTURE SYSTEMS AND METHODS

(71) Applicant: KNOWN MEDICINE INC., Salt Lake City, UT (US)

(72) Inventors: Andrea MAZZOCCHI, Salt Lake City, UT (US); Ryan NEELY, Salt Lake City, UT (US); Katie-Rose SKELLY, Salt Lake City, UT (US); Elizabeth MITCHELL, Salt Lake City, UT (US)

(21) Appl. No.: 18/837,548

(22) PCT Filed: Feb. 13, 2023
(86) PCT No.: PCT/US2023/062505

§ 371 Date: Aug. 9, 2024

Related U.S. Application Data
(60) Provisional application No. 63/309,950, filed on Feb. 14, 2022.

(51) Int. Cl. C12N 5/00

(52) U.S. Cl. CPC C12N 5/0062; C12N 2533/30; C12N 2533/52; C12N 2537/10

(57) ABSTRACT

Disclosed herein is a high-throughput three-dimensional cell-culture system and related methods. The cell-culture system is configured to provide a relatively even distribution of cells across the surface of a well for improved imaging analysis

### CROSS-REFERENCE

This application claims the benefit of U.S. Provisional Application No. 63/309,950, filed Feb. 14, 2022, which application is incorporated herein by reference.

### BACKGROUND OF THE INVENTION

Three-dimensional (3D) cell culture is a promising technology for applications in the development and identification of therapeutic treatments. Relative to two-dimensional cell culture systems, three-dimensional cell culture can have the benefit of more closely mimicking biological function. High quality cell sample preparations are important for various clinical and research applications. 3-D cell cultures can closely represent in vivo characteristics. There is a need for high-throughput 3D cell-culture methods and systems that are capable of effectively and reproducibly culturing cells at a suitable cost.

### SUMMARY OF THE INVENTION

Disclosed herein is a method for producing a three-dimensional hydrogel system in a well, the method comprising: (a) delivering a first mixture comprising a first acrylate and a first thiolated polymer into a well, thereby forming a concave meniscus within the well; (b) cross-linking the first acrylate and the first thiolated polymer of the first mixture to form a first cured hydrogel layer, wherein the first cured hydrogel layer comprises a concave upper surface; (c) delivering a second mixture comprising a second acrylate and a second thiolated polymer into the well, on top of the first cured hydrogel layer; (d) cross-linking the second acrylate and the second thiolated polymer of the second mixture to form a second cured hydrogel layer on top of the first cured hydrogel layer, wherein the second cured hydrogel layer comprises an upper surface that is less concave than the concave upper surface of the first cured hydrogel layer. The upper surface of the second cured layer can be substantially planar.

Crosslinking the first acrylate and the first thiolated polymer to form the first cured hydrogel layer can comprise exposing the first mixture to ultraviolet light while the first mixture is disposed within (positioned within) the well and before the second mixture has been delivered to the well. Crosslinking the second acrylate and the second thiolated polymer to form the second cured hydrogel layer can comprise exposing the second mixture to ultraviolet light while both the first cured hydrogel layer and the second mixture are disposed within the well. The first mixture can be exposed to ultraviolet light for a period of time that is longer than a period of time to which the second mixture is exposed to ultraviolet light. The second mixture can be exposed to ultraviolet light for less than 12 seconds and the first mixture is exposed to ultraviolet light for greater than 12 seconds. In some embodiments, the second mixture can be exposed to ultraviolet light for less than 15 seconds and the first mixture is exposed to ultraviolet light for greater than 15 seconds. In some embodiments, the second mixture and the first mixture are each exposed to ultraviolet light from 5 seconds to 20 seconds (e.g., from 10 second to 20 seconds). The ultraviolet light can have a wavelength from 320 nm and 390 nm. The ultraviolet light can have an intensity of 250 mW/cm².

The second mixture can further comprise a plurality of cells. The plurality of cells can be unaggregated cells. The plurality of cells may be stained prior to (c). The plurality of cells that have been stained can be stained with one or more of (i) a dye specific to cell nuclei, (ii) a dye specific to cell membranes, (iii) a dye specific to live cells, and (iv) a dye specific to dead cells. The plurality of cells can be stained with one or more of a lipophilic carbocyanine dye, a biz-benzimide, a xanthene dye, or propidium iodide. The plurality of cells can be marked with a fluorescent tag prior to delivering the second mixture to the well.

The second mixture can optionally or further comprise laminin such that laminin is non-covalently distributed within the second cured hydrogel. In some embodiments, no laminin is included in the second mixture. The first mixture can further comprise a photoinitiator. The photoinitiator can comprise 2-Hydroxy-4′-(2-hydroxyethoxy)-2-methylpropiophenone. The second mixture can further comprise a photoinitiator. The photoinitiator can comprise 2-Hydroxy-4′-(2-hydroxyethoxy)-2-methylpropiophenone.

The method can further comprise adding cell culture media to the three-dimensional hydrogel system. The cell culture media can comprise one or more of Sodium Phosphate, Potassium Chloride, Sodium Bicarbonate, Ferric Nitrate, Calcium Chloride, or Potassium Chloride. The kinematic viscosity of the first mixture, when delivered to the well, can be between 0.5 mm²/s to 10 mm²/s. The kinematic viscosity of the second mixture, when delivered to the well, can be between 0.5 mm²/s to 10 mm²/s.

One or both of the first acrylate and the second acrylate can be a multi-armed PEG acrylate. One or both of the first acrylate and the second acrylate can be PEG diacrylate. The PEG diacrylate of the first mixture can have a number average molecular weight of greater than three kilodaltons. In some embodiments, the PEG diacrylate of the first mixture can have a number average molecular weight of between three and four kilodaltons. In some embodiments, the PEG diacrylate of the first mixture can have a number average molecular weight of between about 7 and 15 kilodaltons. The PEG diacrylate of the second mixture can have a number average molecular weight of greater than three kilodaltons. In some embodiments, the PEG diacrylate of the second mixture can have a number average molecular weight of between three and four kilodaltons. In some embodiments, the PEG diacrylate of the second mixture can have a number average molecular weight of between 7 and 15 kilodaltons.

One or both of the first thiolated polymer and the second thiolated polymer can be thiolated gelatin. The thiolated gelatin can have a degree of thiolation between 20% and 30%. In some embodiments, the weight ratio of PEG diacrylate to thiolated gelatin in the second mixture is between 1:4 and 2:1, such as 1:2. In some embodiments, the weight ratio of PEG diacrylate to thiolated gelatin in the second mixture is between 1:1 and 8:1 (e.g., between 2:1 and 8:1). The second mixture can comprise 2-Hydroxy-4′-(2-hydroxyethoxy)-2-methylpropiophenone at a weight ratio of 2-Hydroxy-4′-(2-hydroxyethoxy)-2-methylpropiophenone to thiolated gelatin of between 1:20 and 1:100, such as 1:50.

The second mixture can comprise laminin at a weight ratio of thiolated gelatin to laminin of between 1:1 and 8:1, such as 4:1. The concentration of laminin in the second mixture can be at least 0.1 mg/mL. The concentration of laminin in the second mixture can be no more than 1.0 mg/ml . . . concentration of laminin the second mixture can be between 0.2 and 1.0 mg/mL or between 0.2 and 0.6 mg/mL.

The well can be one well of an array of wells of a multi-well plate. The array of wells can comprise at least 96 wells. The array of wells can comprise at least 384 wells. The array of wells can comprise at least 1536 wells. The delivery of the first and second mixtures can be completed over 384 wells in less than one hour. The delivery of the first mixture can be completed over 384 wells in less than 20 minutes. The delivery of the second mixture can be completed over 384 wells in less than 20 minutes. The delivery of the first and second mixtures can be completed over 1536 wells in less than one hour. The delivery of the first mixture can be completed over 1536 wells in less than 30 minutes. The delivery of the second mixture can be completed over 1536 wells in less than 30 minutes.

Delivery of the first mixture can comprise delivery via a robotics system. Delivery of the second mixture can comprise delivery via a robotics system. Delivering the first mixture can comprise dispensing a first amount of the first mixture to the well and aspirating a second amount of the first mixture from the well. The first amount can be greater than the second amount. Delivering the second mixture can comprise dispensing a first amount of the second mixture to the well and aspirating a second amount of the second mixture from the well. The first amount can be greater than the second amount.

The second mixture can comprise cells. The cells can be substantially evenly distributed throughout the second cured hydrogel layer. Disclosed herein is a hydrogel formed by the method described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a single layer of hydrogel with cells concentrated at the walls of the well.

FIG. 1B shows a double layer of hydrogel with cells evenly dispersed across the area of the well.

FIG. 2A is a graph illustrating the growth of HCT-116 cells in gels made from Gelin-S and Ac-PEG-Ac.

FIG. 2B is a graph illustrating a viability comparison between Gelin-S based gels and RGD-based gels.

FIG. 2C is a graph illustrating a viability comparison between a Gelin-S based gel, an RGD based gel, and a pH-balanced RGD gel.

FIGS. 3A and 3B are cross-sectional views of wells 100, 200 containing hydrogel components. Specifically, FIG. 3A depicts a well 100 that has been filled with two hydrogel layers, with the first layer 110 having a more concave upper surface 111 than the upper surface 121 of the second layer 120 that has been deposited on the first layer 110. FIG. 3B depicts a well 200 that has been filled with same total volume of hydrogel as in FIG. 3A, but the well 200 was formed by depositing only a single layer 210. The upper surface 211 of this single layer 210 is more concave than the upper surface 111 of the second layer 110 of the analogous two-layer structure shown in FIG. 3A.

FIG. 4 shows a comparison of cell count analysis between a single hydrogel layer deposition (FIG. 4A) and a two-layer hydrogel layer deposition (FIG. 4B).

FIG. 5 shows an example of a computer system configured to implement methods provided herein.

### DETAILED DESCRIPTION OF THE INVENTION

Many hydrogel-forming mixtures are unduly viscous and spontaneously crosslink into gel form on short timescales (for example, minutes) at room temperatures. This creates challenges for automation, which often relies on liquid handling robots that have trouble handling viscous liquids and working with materials with inconsistent properties. There is a need for a hydrogel manufacturing methods in which hydrogel-forming mixtures remain in a relatively low-viscosity form to dispense the hydrogel into a high-throughput-compatible array of wells prior to cross-linking while simultaneously maintaining a relatively even dispersion of cells within the hydrogel system or a portion thereof.

Disclosed herein are systems and methods of high-throughput, three-dimensional cell culturing. Hydrogel-forming mixtures can naturally wick up the side of a well, concentrating the hydrogel (and cells within the hydrogel) on the outside walls (e.g., the periphery) of the well. Methods and systems described herein are configured to account for a propensity of hydrogel-forming mixtures to concentrate the hydrogel (and cells within it) on or adjacent to the outside walls of the well by using a two-layer deposition process that allows for robust growth of cells in the center of the well (and along the periphery of the well). Thus, the cells of the present disclosure may be more evenly dispersed across the area of the well. The manufacturing method can comprises curing a first layer of hydrogel in a well to create a concave upper surface. The curing process can involve a photo-initiator. Curing can comprise light exposure (e.g., exposure to UV light). The method can comprise depositing and curing a second layer of hydrogel comprising the cells on top of the first layer. The second layer can comprise one or more proteins (e.g., laminin) configured to interact with the cells, such as mammalian cells. The first layer and the second layer can have the same refractive index after curing. The porosity of the hydrogel can be configured to be large enough to allow diffusion of nutrients and dyes, but small enough to retain cells, such as immune cells.

### Thiolated Polymer

A hydrogel (of hydrogel layer) can comprise and/or be formed from a thiolated polymer. The thiolated polymer can be gelatin or poly-ethylene glycol (PEG). The thiolated polymer can have a number average molecular weight greater than 1 kDa, 1.5 kDa, 2 kDa, 2.5 kDa, 3 kDa, 3.5 kDa, 4 kDa, 4.5 kDa, 5 kDa, 5.5 kDa, 6 kDa, 6.5 kDa, 7 kDa, 7.5 kDa, 8 kDa, 8.5 kDa, 9 kDa, 9.5 kDa, 10 kDa, 10.5 kDa, 11 kDa, 11.5 kDa, 12 kDa, 12.5 kDa, 13 kDa, 13.5 kDa, 14 kDa, 14.5 kDa, 15 kDa, 15.5 kDa, 16 kDa, 16.5 kDa, 17 kDa, 17.5 kDa, 18 kDa, 18.5 kDa, 19 kDa, 19.5 kDa, 20 kDa, 20.5 kDa, 21 kDa, 21.5 kDa, 22 kDa, 22.5 kDa, 23 kDa, 23.5 kDa, 24 kDa, 24.5 kDa, 25 kDa, 25.5 kDa, 26 kDa, 26.5 kDa, 27 kDa, 27.5 kDa, 28 kDa, 28.5 kDa, 29 kDa, 29.5 kDa, 30 kDa, 30.5 kDa, 31 kDa, 31.5 kDa, 32 kDa, 32.5 kDa, 33 kDa, 33.5 kDa, 34 kDa, 34.5 kDa, or 35 kDa. The thiolated polymer can have a number average weight less than 35 kDa, 34.5 kDa, 34 kDa, 33.5 kDa, 33 kDa, 32.5 kDa, 32 kDa, 31.5 kDa, 31 kDa, 30.5 kDa, 30 kDa, 29.5 kDa, 29 kDa, 28.5 kDa, 28 kDa, 27.5 kDa, 27 kDa, 26.5 kDa, 26 kDa, 25.5 kDa, 25 kDa, 24.5 kDa, 24 kDa, 23.5 kDa, 23 kDa, 22.5 kDa, 22 kDa, 21.5 kDa, 21 kDa, 20.5 kDa, 20 kDa, 19.5 kDa, 19 kDa, 18.5 kDa, 18 kDa, 17.5 kDa, 17 kDa, 16.5 kDa, 16 kDa, 15.5 kDa, 15 kDa, 14.5 kDa, 14 kDa, 13.5 kDa, 13 kDa, 12.5 kDa, 12 kDa, 11.5 kDa, 11 kDa, 10.5 kDa, 10 kDa, 9.5 kDa, 9 kDa, 8.5 kDa, 8 kDa, 7.5 kDa, 7 kDa, 6.5 kDa, 6 kDa, 5.5 kDa, 5 kDa, 4.5 kDa, 4 kDa, 3.5 kDa, 3 kDa. 2.5 kDa, 2 kDa, 1.5 kDa, or 1 kDa. The thiolated polymer can have a number average molecular weight from 1 kDa to 2 kDa, from 1.5 kDa to 2.5 kDa, from 2 kDa to 3 kDa, from 2.5 kDa to 3.5 kDa, from 3 kDa to 4 kDa, from 3.5 kDa to 4.5 kDa, from 4 kDa to 5 kDa, from 4.5 kDa to 5.5 kDa, from 5 kDa to 6 kDa, from 5.5 kDa to 6.5 kDa, from 7 kDa to 8 kDa, from 7.5 kDa to 8.5 kDa, from 9 kDa to 10 kDa, from 9.5 kDa to 10.5 kDa, from 10 kDa to 11 kDa, from 10.5 kDa to 11.5 kDa, from 11 kDa to 12 kDa, from 11.5 kDa to 12.5 kDa, from 12 kDa to 13 kDa, from 12.5 kDa to 13.5 kDa, from 13 kDa to 14 kDa, from 13.5 kDa to 14.5 kDa, from 14 kDa to 15 kDa, from 14.5 kDa to 15.5 kDa, from 15 kDa to 16 kDa, from 15.5 kDa to 16.5 kDa, from 16 kDa to 17 kDa, from 16.5 kDa to 17.5 kDa, from 17 kDa to 18 kDa, from 17.5 kDa to 18.5 kDa, from 18 kDa to 19 kDa, from 18.5 kDa to 19.5 kDa, from 19 kDa to 20 kDa, from 19.5 kDa to 20.5 kDa, from 20 kDa to 21 kDa, from 20.5 kDa to 21.5 kDa, from 21 kDa to 22 kDa, from 21.5 kDa to 22.5 kDa, from 22 kDa to 23 kDa, from 22.5 kDa to 23.5 kDa, from 23 kDa to 24 kDa, from 23.5 kDa to 24.5 kDa, from 24 kDa to 25 kDa, from 24.5 kDa to 25.5 kDa, from 25 kDa to 26 kDa, from 25.5 kDa to 26.5 kDa, from 26 kDa to 27 kDa, from 26.5 kDa to 27.5 kDa, from 27 kDa to 28 kDa, from 27.5 kDa to 28.5 kDa, from 28 kDa to 29 kDa, from 28.5 kDa to 29.5 kDa, from 29 kDa to 30 kDa, from 29.5 kDa to 30.5 kDa, from 30 kDa to 31 kDa, from 30.5 kDa to 31.5 kDa, from 31 kDa to 32 kDa, from 31.5 kDa to 32.5 kDa, from 32 kDa to 33 kDa, from 32.5 kDa to 33.5 kDa, from 33 kDa to 34 kDa, from 33.5 kDa to 34.5 kDa, from 34 kDa to 35 kDa.

The thiolated polymer can have a degree of thiolation greater than 0.5%, 1%, 2%, 3%, 4%, 5%, 6%, 7%, 8%, 9%, 10%, 15%, 20%, 25%, 30%, 35%, 40%, 45%, or 50%. The thiolated polymer can have a degree of thiolation less than 50%, 45%, 40%, 35%, 30%, 25%, 20%, 15%, 10%, or 5%. The thiolated polymer can have a degree of thiolation from 5% to 15%, from 10% to 20%, from 15% to 25%, from 20% to 30%, from 30% to 40%, or from 35% to 45%.

Degree of thiolation as described herein can refer to a percentage of free (e.g., unreacted) sulfhydryl groups (e.g., “thiol groups”, —SH groups) out of a total number of sulfhydryl groups on a molecule. Free sulfhydryl groups can be measured using any technique known by one of skill in the art, for example, Ellman's assay. Ellman's assay uses 5.5′-dithio-bis-(2-nitrobenzoic acid) (DTNB) to react with free sulfhydryl groups to yield a mixed disulfide and 2-nitro-5-thiobenzoic acid (TNB). TNB has a high molar extinction coefficient in the visible range and therefore can be directly related to a total number of free sulfhydryl groups in solution.

The thiolated PEG can have a number average molecular weight greater than 1 kDa, 1.5 kDa, 2 kDa, 2.5 kDa, 3 kDa, 3.5 kDa, 4 kDa, 4.5 kDa, 5 kDa, 5.5 kDa, 6 kDa, 6.5 kDa, 7 kDa, 7.5 kDa, 8 kDa, 8.5 kDa, 9 kDa, 9.5 kDa, or 10 kDa. The thiolated PEG can have an average weight less than 10 kDa, 9.5 kDa, 9 kDa, 8.5 kDa, 8 kDa, 7.5 kDa, 7 kDa, 6.5 kDa, 6 kDa, 5.5 kDa, 5 kDa, 4.5 kDa, 4 kDa, 3.5 kDa, 3 kDa. 2.5 kDa, 2 kDa, 1.5 kDa, or 1 kDa. The thiolated PEG can have a number average molecular weight from 1 kDa to 2 kDa, from 1.5 kDa to 2.5 kDa, from 2 kDa to 3 kDa, from 2.5 kDa to 3.5 kDa, from 3 kDa to 4 kDa, from 3.5 kDa to 4.5 kDa, from 4 kDa to 5 kDa, from 4.5 kDa to 5.5 kDa, from 5 kDa to 6 kDa, from 5.5 kDa to 6.5 kDa, from 7 kDa to 8 kDa, from 7.5 kDa to 8.5 kDa, or from 9 kDa to 10 kDa.

The thiolated gelatin can have a number average molecular weight greater than 1 kDa, 1.5 kDa, 2 kDa, 2.5 kDa, 3 kDa, 3.5 kDa, 4 kDa, 4.5 kDa, 5 kDa, 5.5 kDa, 6 kDa, 6.5 kDa, 7 kDa, 7.5 kDa, 8 kDa, 8.5 kDa, 9 kDa, 9.5 kDa, 10 kDa, 10.5 kDa, 11 kDa, 11.5 kDa, 12 kDa, 12.5 kDa, 13 kDa, 13.5 kDa, 14 kDa, 14.5 kDa, 15 kDa, 15.5 kDa, 16 kDa, 16.5 kDa, 17 kDa, 17.5 kDa, 18 kDa, 18.5 kDa, 19 kDa, 19.5 kDa, 20 kDa, 20.5 kDa, 21 kDa, 21.5 kDa, 22 kDa, 22.5 kDa, 23 kDa, 23.5 kDa, 24 kDa, 24.5 kDa, 25 kDa, 25.5 kDa, 26 kDa, 26.5 kDa, 27 kDa, 27.5 kDa, 28 kDa, 28.5 kDa, 29 kDa, 29.5 kDa, 30 kDa, 30.5 kDa, 31 kDa, 31.5 kDa, 32 kDa, 32.5 kDa, 33 kDa, 33.5 kDa, 34 kDa, 34.5 kDa, or 35 kDa. The thiolated gelatin can have a number average weight less than 35 kDa, 34.5 kDa, 34 kDa, 33.5 kDa, 33 kDa, 32.5 kDa, 32 kDa, 31.5 kDa, 31 kDa, 30.5 kDa, 30 kDa, 29.5 kDa, 29 kDa, 28.5 kDa, 28 kDa, 27.5 kDa, 27 kDa, 26.5 kDa, 26 kDa, 25.5 kDa, 25 kDa, 24.5k Da, 24 kDa, 23.5 kDa, 23 kDa, 22.5 kDa, 22 kDa, 21.5 kDa, 21 kDa, 20.5 kDa, 20 kDa, 19.5 kDa, 19 kDa, 18.5 kDa, 18 kDa, 17.5 kDa, 17 kDa, 16.5 kDa, 16 kDa, 15.5 kDa, 15 kDa, 14.5 kDa, 14 kDa, 13.5 kDa, 13 kDa, 12.5 kDa, 12 kDa, 11.5 kDa. 11 kDa, 10.5 kDa, 10 kDa, 9.5 kDa, 9 kDa, 8.5 kDa, 8 kDa, 7.5 kDa, 7 kDa, 6.5 kDa, 6 kDa, 5.5 kDa, 5 kDa, 4.5 kDa, 4 kDa, 3.5 kDa, 3 kDa. 2.5 kDa, 2 kDa, 1.5 kDa, or 1 kDa. The thiolated gelatin can have a number average molecular weight from 1 kDa to 2 kDa, from 1.5 kDa to 2.5 kDa, from 2 kDa to 3 kDa, from 2.5 kDa to 3.5 kDa, from 3 kDa to 4 kDa, from 3.5 kDa to 4.5 kDa, from 4 kDa to 5 kDa, from 4.5 kDa to 5.5 kDa, from 5 kDa to 6 kDa, from 5.5 kDa to 6.5 kDa, from 7 kDa to 8 kDa, from 7.5 kDa to 8.5 kDa, from 9 kDa to 10 kDa, from 9.5 kDa to 10.5 kDa, from 10 kDa to 11 kDa, from 10.5 kDa to 11.5 kDa, from 11 kDa to 12 kDa, from 11.5 kDa to 12.5 kDa, from 12 kDa to 13 kDa, from 12.5 kDa to 13.5 kDa, from 13 kDa to 14 kDa, from 13.5 kDa to 14.5 kDa, from 14 kDa to 15 kDa, from 14.5 kDa to 15.5 kDa, from 15 kDa to 16 kDa, from 15.5 kDa to 16.5 kDa, from 16 kDa to 17 kDa, from 16.5 kDa to 17.5 kDa, from 17 kDa to 18 kDa, from 17.5 kDa to 18.5 kDa, from 18 kDa to 19 kDa, from 18.5 kDa to 19.5 kDa, from 19 kDa to 20 kDa, from 19.5 kDa to 20.5 kDa, from 20 kDa to 21 kDa, from 20.5 kDa to 21.5 kDa, from 21 kDa to 22 kDa, from 21.5 kDa to 22.5 kDa, from 22 kDa to 23 kDa, from 22.5 kDa to 23.5 kDa, from 23 kDa to 24 kDa, from 23.5 kDa to 24.5 kDa, from 24 kDa to 25 kDa, from 24.5 kDa to 25.5 kDa, from 25 kDa to 26 kDa, from 25.5 kDa to 26.5 kDa, from 26 kDa to 27 kDa, from 26.5 kDa to 27.5 kDa, from 27 kDa to 28 kDa, from 27.5 kDa to 28.5 kDa, from 28 kDa to 29 kDa, from 28.5 kDa to 29.5 kDa, from 29 kDa to 30 kDa, from 29.5 kDa to 30.5 kDa, from 30 kDa to 31 kDa, from 30.5 kDa to 31.5 kDa, from 31 kDa to 32 kDa, from 31.5 kDa to 32.5 kDa, from 32 kDa to 33 kDa, from 32.5 kDa to 33.5 kDa, from 33 kDa to 34 kDa, from 33.5 kDa to 34.5 kDa, from 34 kDa to 35 kDa.

### Acrylate

A hydrogel (of hydrogel layer) can comprise and/or be formed from an acrylate. The acrylate can comprise a poly-ethylene glycol (PEG) with one or more acrylate groups. In some embodiments, the PEG acrylate can be a multi-armed PEG acrylate. In some embodiments, the acrylate can be PEG diacrylate. The PEG acrylate (e.g., PEG diacrylate) can have a number average molecular weight greater than 1 kDa, 1.5 kDa, 2 kDa, 2.5 kDa, 3 kDa, 3.5 kDa, 4 kDa, 4.5 kDa, 5 kDa, 5.5 kDa, 6 kDa, 6.5 kDa, 7 kDa, 7.5 kDa, 8 kDa, 8.5 kDa, 9 kDa, 9.5 kDa, or 10 kDa. The PEG can have a number average weight less than 10 kDa, 9.5 kDa, 9 kDa, 8.5 kDa, 8 kDa, 7.5 kDa, 7 kDa, 6.5 kDa, 6 kDa, 5.5 kDa, 5 kDa, 4.5 kDa, 4 kDa, 3.5 kDa, 3 kDa. 2.5 kDa, 2 kDa, 1.5 kDa, or 1 kDa. The PEG acrylate can have a number average molecular weight from 1 kDa to 2 kDa, from 1.5 kDa to 2.5 kDa, from 2 kDa to 3 kDa, from 2.5 kDa to 3.5 kDa, from 3 kDa to 4 kDa, from 3.5 kDa to 4.5 kDa, from 4 kDa to 5 kDa, from 4.5 kDa to 5.5 kDa, from 5 kDa to 6 kDa, from 5.5 kDa to 6.5 kDa, from 7 kDa to 8 kDa, from 7.5 kDa to 8.5 kDa, or from 9 kDa to 10 kDa.

### Photoinitiator

The acrylate and the thiolated polymer can be cross-linked using a photoinitiator. The photo-initiator can comprise 2-Hydroxy-4′-(2-hydroxyethoxy)-2-methylpropiophenone (Irgacure 2959).

The photoinitiator can be a cationic or radical photoinitiator. Radical photoinitiators are water-compatible and act on molecules containing an acrylate or styrene group. The photoinitiator can comprise Irgacure 2959, 184, or 651.

### Hydrogel Curing Conditions

A first or second hydrogel layer can be cured using light (e.g., UV light) having an average wavelength of less than 400 nm, 390 nm, 380 nm, 370 nm, 360 nm, 350 nm, 340 nm, 330 nm, 320 nm, or 310 nm. The first or second hydrogel layer can be cured using light having an average wavelength of at least 310 nm, 320 nm, 330 nm, 340 nm, 350 nm, 360 nm, 370 nm, 380 nm, 390 nm, or 400 nm. The first or second hydrogel layer can be cured using light having an average wavelength of from 300 nm-320 nm, 310 nm-330 nm, 320 nm-340 nm, 330 nm-350 nm, 340 nm-360 nm, 350 nm-370 nm, 360 nm-380 nm, 370 nm-390 nm, or 380 nm-400 nm.

The first hydrogel layer can be cured for greater than 5 seconds, 10 seconds, 15 seconds, 20 seconds, 25 seconds, or 30 seconds. The first hydrogel layer can be cured for less than 60 seconds, 50 second, 40 seconds, 30 seconds, 20 seconds, or 10 seconds. The first hydrogel layer can be cured for between 5 seconds and 15 seconds, 10 seconds and 20 seconds, 15 seconds and 25 seconds, 20 seconds and 30 seconds, 25 seconds and 35 seconds, 30 seconds and 40 seconds, 35 seconds and 45 seconds, or 40 seconds and 50 seconds.

The second hydrogel layer can be cured for greater than 1 second, 3 seconds, 5 seconds, 8 seconds, or 10 seconds. The second hydrogel layer can be cured for less than 15 seconds, 10 seconds, 8 seconds, 5 seconds, or 3 seconds. The second hydrogel layer can be cured for between 1 second and 5 seconds, 3 seconds and 8 seconds, 5 seconds and 10 seconds, or 8 seconds and 15 seconds.

### Media

A cell culture comprising a hydrogel system can comprise media. The media can comprise one or more of sodium phosphate, potassium chloride, sodium bicarbonate, ferric nitrate, calcium chloride, or potassium chloride. The media can comprise any suitable media known in the art. Suitable culture media can include, for example, Dulbecco's Phosphate Buffered Saline, Earle's Balanced Salts, Hanks' Balanced Salts, Tyrode's Salts, Alsever's Solution, Gey's Balanced Salt Solution, Kreb's-Henseleit Buffer Modified, Kreb's-Ringer Bicarbonate Buffer, Puck's Saline, Dulbecco's Modified Eagle's Medium, Dulbecco's Modified Eagle's Medium/Nutrient F-12 Ham, Nutrient Mixture F-10 Ham (Ham's F-10), Medium 199, Minimum Essential Medium Fagle, RPMI-1640 Medium, Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E, or combinations thereof. The media can be modified or supplemented. The media can further comprise albumin, selenium, transferrins, fetuins, sugars, amino acids, vitamins, growth factors, cytokines, hormones, antibiotics, lipids, lipid carriers, cyclodextrins, or a combination thereof.

### Cell Types

Cells can be cultured, grown or maintained in a culture system that comprises a hydrogel layer of hydrogel system that includes multiple (e.g., two) hydrogel layers. The cells can be eukaryotic cells or prokaryotic cells. In some embodiments, the cells are mammalian cells or plant cells. In some embodiments, the cells are derived from a tumor. The cells can be derived from a tissue. The cells can be liver cells, gastrointestinal cells, lymphoid cells, pancreatic cells, kidney cells, lung cells, tracheal cells, vascular cells, skeletal muscle cells, cardiac cells, skin cells, smooth muscle cells, connective tissue cells, corneal cells, genitourinary cells, breast cells, reproductive cells, endothelial cells, epithelial cells, fibroblasts, neural cells, Schwann cells, adipose cells, bone cells, bone marrow cells, cartilage cells, pericytes, mesothelial cells, stromal cells, stem cells, progenitor cells, lymph cells, blood cells, endoderm-derived cells, ectoderm-derived cells, or mesoderm-derived cells. The cells can comprise T-cell, B-cells, monocytes, macrophages, dendritic cells, eosinophils, basophils, mast cells, or natural killer cells. The cells can be obtained, for example, by resection, biopsy, fine needle aspirations, brushing, or swab.

### Cell Labeling

The cells can be labeled with a stain or marker. The cells can be labeled with a dye specific to cell nuclei, a dye specific to cell membranes, a dye specific to live cells, a dye specific to dead cells, or a fluorescent tag. The fluorescent tag can be attached to an antibody or derivative thereof. The cells can be labeled with one or more of a lipophilic carbocyanine dye, a biz-benzimide, a xanthene dye, or propidium iodide. The cells can be labeled with, for example, Hoeschst 33342, CellBrite NIR 680, Calcein-AM, or Live-or-Dye NucFix Red. In some embodiments, the cells are stained, labeled, or marked prior to being introduced into a hydrogel system. In other embodiments, the cells are stained after being introduced into a hydrogel system.

### Extracellular Matrix Components

One or more hydrogel layers can comprise one or more component of an extracellular matrix or derivatives thereof. For instance, a second hydrogel layer (e.g., a layer delivered on top of a first layer) can comprise one or more components of an extracellular matrix or derivatives thereof. The components can comprise proteins produced by cells that hold tissues together, provide tensile strength, or facilitate cell signaling. The extracellular matrix components can comprise collagen, fibronectin, laminin, hyaluronates, elastin, or proteoglycans. The extracellular matrix components can be purified from a human or animal source, or produced by recombinant methods known in the art.

### Hydrogel

The hydrogel can comprise one or more (or all of) (1) a PEG diacrylate, (2) one or more thiolated polymers (e.g., thiolated gelatin or thiolated PEG), (3) water, (4) laminin, and (5) irgacure. The hydrogel of hydrogel system can comprise at least two layers. The weight ratio of acrylate to thiolated polymer in each layer can be the same. The acrylate can be a PEG diacrylate. The thiolated polymer can be a thiolated PEG or a thiolated gelatin.

Before curing, the kinematic viscosity of the mixture of thiolated polymer and acrylate can be between 0.5 mm²/s to 10 mm²/s. Before curing, the kinematic viscosity of the first and/or the second mixture of thiolated polymer and acrylate can be between 0.5 mm²/s to 10 mm²/s. The kinematic viscosity of the mixture can be greater than 0.1 mm²/s, 0.5 mm²/s, 1.0 mm²/s, 1.5 mm²/s, 2.0 mm²/s, 2.5 mm²/s, 3.0 mm²/s, 3.5 mm²/s, 4.0 mm²/s, 4.5 mm²/s, 5.0 mm²/s, 5.5 mm²/s, 6.0 mm²/s, 6.5 mm²/s, 7.0 mm²/s, 7.5 mm²/s, 8.0 mm²/s, 8.5 mm²/s, 9.0 mm²/s, or 9.5 mm²/s. The kinematic viscosity of the mixture can be less than 10.0 mm²/s, 9.5 mm²/s, 9.0 mm²/s, 8.5 mm²/s, 8.0 mm²/s, 7.5 mm²/s, 7.0 mm²/s, 6.5 mm²/s, 6.0 mm²/s, 5.5 mm²/s, 5.0 mm²/s, 4.5 mm²/s, 4.0 mm²/s, 3.5 mm²/s, 3.0 mm²/s, 2.5 mm²/s, 2.0 mm²/s, 1.5 mm²/s, 1.0 mm²/s, or 0.5 mm²/s. The kinematic viscosity of the mixture can be from 0.5 mm²/s to 1.5 mm²/s, 1.0 mm²/s to 2.0 mm²/s, 1.5 mm²/s to 2.5 mm²/s, 2.0 mm²/s to 3.0 mm²/s, 2.5 mm²/s to 3.5 mm²/s, 3.0 mm²/s to 4.0 mm²/s, 3.5 mm²/s to 4.5 mm²/s, 4.0 mm²/s to 5.0 mm²/s, 4.5 mm²/s to 5.5 mm²/s, 5.0 mm²/s to 6.0 mm²/s, 5.5 mm²/s to 6.5 mm²/s, 6.0 mm²/s to 7.0 mm²/s, 6.5 mm²/s to 7.5 mm²/s, 7.0 mm²/s to 8.0 mm²/s, 7.5 mm²/s to 8.5 mm²/s, 8.0 mm²/s to 9.0 mm²/s, 8.5 mm²/s to 9.5 mm²/s, or 9.0 mm²/s to 10.0 mm²/s.

The weight ratio of acrylate to thiolated polymer in the hydrogel or hydrogel system can be greater than 1:1 or 1:2. The weight ratio of the acrylate to the thiolated polymer in the first layer or the second layer can be less than 1:2 or 1:1. The weight ratio of PEG diacrylate to thiolated gelatin can be 1:2. The weight ration of PEG diacrylate to thiolated gelatin can be between 1:4 and 2:1.

The weight ratio of thiolated polymer to photoinitiator in the hydrogel can be 40:1, 45:1, 50:1, 55:1, or 60:1. The weight ratio of 2-Hydroxy-4′-(2-hydroxyethoxy)-2-methylpropiophenone to thiolated gelatin can be between 1:20 to 1:100. The weight ratio of 2-Hydroxy-4′-(2-hydroxyethoxy)-2-methylpropiophenone to thiolated gelatin can be 1:50.

The weight ratio of thiolated polymer to extracellular matrix component can be 3:1, 4:1, or 5:1. The weight ratio of thiolated gelatin to laminin can be 4:1. The concentration of extracellular matrix component can be less than 0.5 mg/mL, 0.4 mg/mL, 0.3 mg/mL, 0.2 mg/mL, or 0.1 mg/mL. The concentration of extracellular matrix component can be at least 0.1 mg/mL, 0.15 mg/mL, 0.20 mg/mL, 0.25 mg/mL, 0.30 mg/mL, 0.35 mg/mL. 0.40 mg/mL, or 0.5 mg/mL.

### Array

A hydrogel or hydrogel system may be disposed within wells of array (e.g., wells of a multi-well plate). The array can comprise a plurality of wells. The well can be a well of a multi-well plate. The array of wells can comprise at least 5, 10, 20, 50, 100, 150, 200, 300, 400, 500, 1000, 2000, 3000, 4000, or 5000 wells. The wells can comprise at least 96 wells, 384 wells, or 1536 wells.

### Uses

The three-dimensional cell-culture systems and methods described herein can be used to identify therapeutics for disease treatment or to identify which tissues are likely to be susceptible to a known therapeutic. The three-dimensional cell-culture systems and methods described herein can be used to identify reagents for use in researching cell interactions and reactions in a three-dimensional culture. The three-dimensional cell-culture systems described herein can be used to create cell aggregates capable of mimicking in-vivo tissue responses to stimuli.

In certain aspects, the methods and systems disclosed herein may be useful for determining a treatment course for a subject. For example, such methods and systems may involve screening compounds to identify a compound that provide a patient-specific or tissue-specific response. The subject providing a tissue or cellular sample can be healthy. The subject can be positive for a disease. The disease can be an infection. The infection can be viral, bacterial, or fungal. The subject can have an auto-immune disease. The subject can be positive for a tissue-specific disease. The methods and systems may involve a screen that can be used to identify a tissue-specific response within a particular population of individuals.

The subject can be positive for a disease. The disease can be a disease of the liver, heart, kidneys, pancreas, colon, skin, brain, thymus, bone, or lung. The disease can be cancer. The disease can be bladder cancer, breast cancer, colon cancer, rectal cancer, endometrial cancer, kidney cancer, liver cancer, lung cancer, melanoma, non-Hodgkin lymphoma, pancreatic cancer, prostate cancer, or thyroid cancer. The cancer can be, for example, melanoma, ovarian cancer, hypopharyngeal cancer, renal cancer, lung cancer, prostate cancer, breast cancer, pancreatic cancer, hepatocellular cancer, or a cancer of the head or neck. The disease can be a bacterial infection, a viral infection, or a fungal infection.

### Kits

A kit may include one or more containers housing one or more of the components provided in this disclosure and instructions for use. Specifically, such kits may include one or more compositions described herein, along with instructions describing the intended application and the proper use and/or disposition of these compositions. Kits may contain the components in appropriate concentrations or quantities for running various experiments.

### Computer Systems

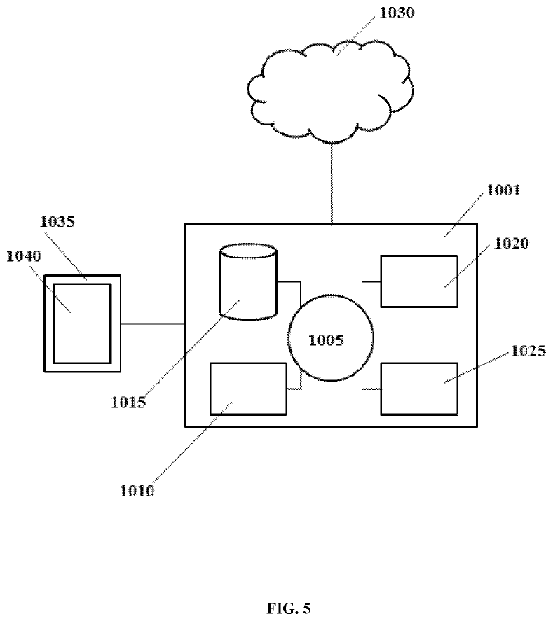

The present disclosure provides computer systems for implementing methods provided herein. FIG. 5 shows an example of a computer system 1001. The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 05 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., remote cloud server). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, for example, shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, an electronic output of identified gene fusions. Examples of UI's include, without limitation, a graphical user interface (GUI) and web-based user interface. The computer system 1001 can include or be in communication with a robotic arm for executing, for example, one or more mixing, depositing, or imaging steps. Examples of robotic arms include, without limitation, a multi-pipette arm, a camera, and a palletizer for moving multi-well plates.

Methods and systems of the present disclosure can be implemented by way of one or more algorithms. An algorithm can be implemented by way of software upon execution by the central processing unit 1005.

### EXAMPLES

### Example 1—Gel Creation

Pre-cured mixtures for forming hydrogels can often naturally wick up the side of the well, forming a concave meniscus within the well. In pre-cured mixtures that include cells, the cells—due to the wicking—can become concentrated on the periphery of the well leaving a central portion of the well that has a relative low cell density. This is less desirable for imaging and promoting cell-cell interactions.

Disclosed herein is a two-stage plating method in which a cell-free mixture is added to a well and then crosslinked within the well to form a first hydrogel layer, leaving a concave cavity (or “basket” of space) in the center of the well. Next, a second mixture that contains cells is added to the well on top of the first hydrogel layer. After curing, the second mixture forms a second cured hydrogel layer that has an upper surface that is less concave than the upper surface of the first hydrogel layer (e.g., an upper surface that is substantially planar). The cells are distributed throughout the second layer, resulting in a relatively even distribution of cells across an x-y plane of the well. Images can be taken using z-stack imaging, in which multiple (e.g., 10) images can be taken of the x-y axis a specified distance (e.g., 100 microns) apart in the z-axis (top to bottom of the well) and subsequently be projected to create a single image.

Mixtures for forming the first and second hydrogel layers were formulated according to the following table:

TABLE 1
Hydrogel Components

| | | | Laminin | |
|---|---|---|---|---|
| | AC-PEG-AC, | Gelin-S, 10% | solution~ | |
| | 3.4 kDa, 10% | dissolved in | 1-2 | |
| | w/w dissolved | an aqueous | mg/mL | Deionized |
| | in an aqueous | solution | (second | water |
| | solution of 0.2% | of 0.1% w/w | layer | (first |
| Component | w/w irgacure | irgacure | only) | layer only) |
| Vol. per mL | 0.25 | 0.5 | 0.25 | 0.25 |

125 mg of the AC-PEG-AC (e.g., Formula I) was added to a 1.5 mL microcentrifuge tube. A 0.2% irgacure solution was added to the tube containing AC-PEG-AC and mixed. A 50 mg bottle of Gelin-S was resuspended in 500 μL of a 0.1% irgacure solution. The AC-PEG-AC solution and the Gelin-S mixture were combined and immediately plated into a 384-well source plate. Immediately after the first layer was plated, it was cured with UV light (320 nm-390 nm) for 15 seconds with an intensity of 250 mW/cm².

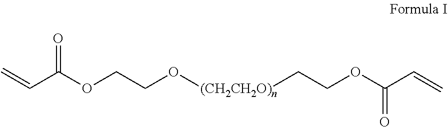

Cells were stained as described in Example 2 and centrifuged at 300×g for 5 minutes to create a cell pellet. The supernatant was removed from the cell pellet.

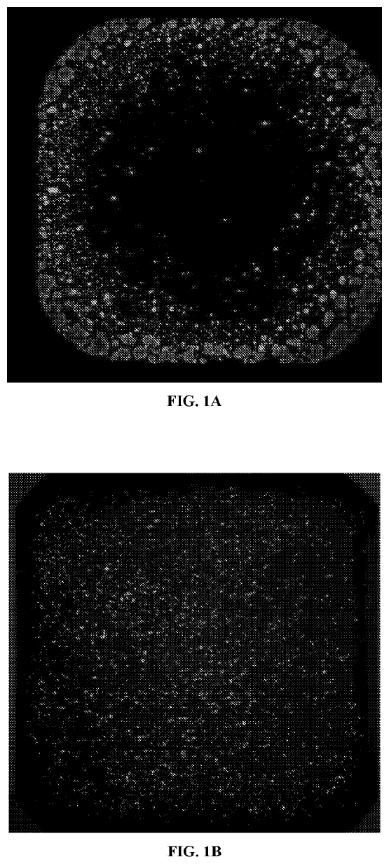

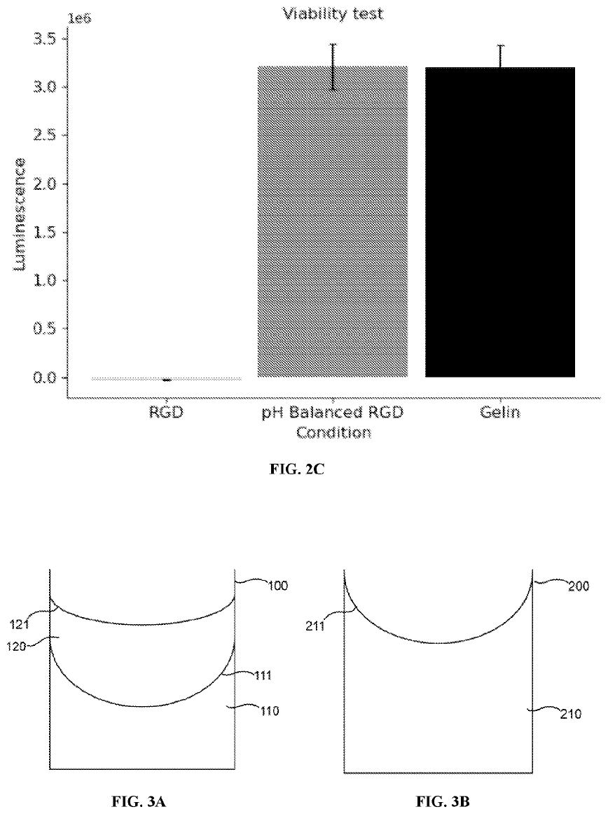

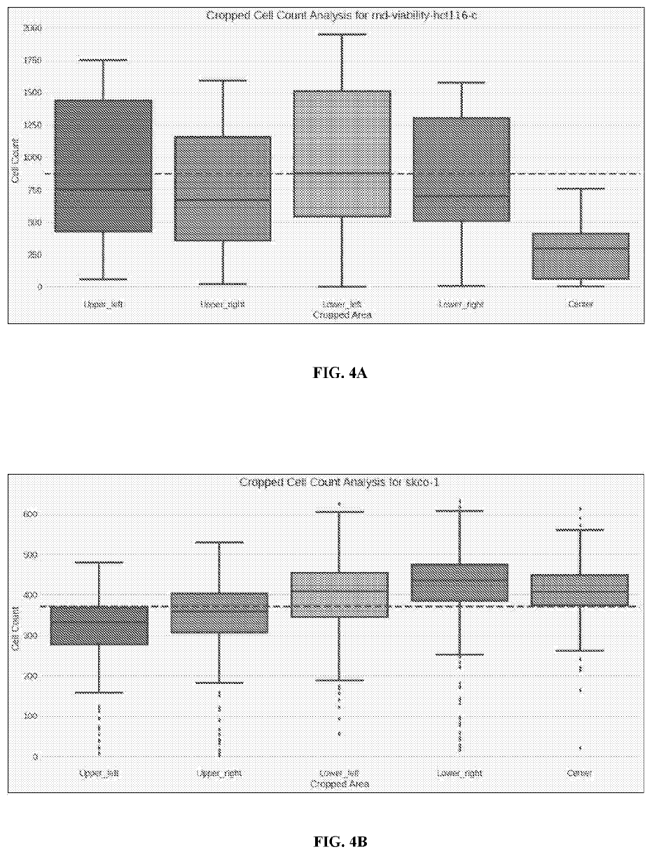

To form a second cured hydrogel layer, the AC-PEG-AC solution, the laminin solution, and the Gelin-S mixture were combined as set forth in Table 1 with the pellet of stained cells. The stained cells were re-suspended in the gel mixture to create a seeded gel mixture. The seeded gel mixture was plated on top of the first layer and immediately cured with UV light (320 nm-390 nm) for 10 seconds with an intensity of 250 mW/cm². At the time that the mixture for the first layer and mixture for second layer were added to the wells, each had a kinematic viscosity of approximately 9.65 mm²/s. As can be seen in FIG. 1B (DAPI channel only), the second layer of stained cells resulted in an even distribution of cells across the well as compared to a single layer of seeded gel in an identical well (FIG. 1A; DAPI channel only). The images were obtained with a confocal microscope using a 4× air objective using a DAPI channel corresponding to the excitation of fluorophore Hoechst. Images can comprise a pseudocomposite of 4 channels: DAPI, Cy5, FITC, and Cy3 corresponding to the excitation of the fluorophores Hoechst, CellTracker Deep Red, Calcein AM, and NucFix red, respectively. For each channel, 10 images were taken 100 microns apart in the Z-axis and projected using the maximum projection to create a single image (e.g., z-stack imaging). The cells throughout each well were counted and the distribution was quantified, as can be seen in FIGS. 4A and 4B wherein FIG. 4B shows the distribution in the two-layer hydrogel formed in this example, while FIG. 4A shows the distribution of cells in an analogous single-layer hydrogel of the same volume as the hydrogel in FIG. 4B, formed from the components from the second layer of the hydrogel as depicted in Table 1. Max-projected images from all wells in each condition (352 per condition) were cropped into 5 equally sized boxes, corresponding to the upper left corner, lower left corner, lower right corner, upper right corner, and center of the image. Cell counts of each cropped image were done using a custom algorithm and counts for all wells were used to create the box-and-whisker plots An example cross-section of a single layer 210 hydrogel FIG. 3B in a well 200 compared to a two-layer hydrogel FIG. 3A in a well 100 can be seen in FIG. 3. While the wells 100, 200 of FIGS. 3A and 3B have the same volume of hydrogel, the upper surface 211 of the single layer 210 is more concave than the upper surface 121 of the second layer of the analogous two-layer structure shown in FIG. 3A. In some embodiments, the upper surface 121 of the second layer is substantially planar.

### Example 2—Cell Staining with Hoechst Nuclear Stain and CellBrite Cytoplasmic Membrane Dye

A T75 flask of HCT-116 cells (colorectal carcinoma cell line (ATCC CCL-247)) was grown at 37° C. for 4 days from last passage in DMEM supplemented with 10% FBS and 1× primocin. The cells were released from the flask bottom using trypsin-EDTA 0.25% by washing the cells twice with 2 mL of trypsin-EDTA and subsequently adding 2 mL of trypsin-EDTA and placing the flask back in the 37° C. incubator for 7 minutes. The cells were then washed into a corner of the flask using 4 mL fresh, warm media. The cells and media were then transferred to a 15 ml conical tube. The conical tube was centrifuged at 300×g for 5 minutes to pellet the cells. The cells were then counted using an automated cell counter. The cells were then stained with Hoechst 3342 (Abcam ab228551; to identify cell nuclei) and Cell Brite NIR 680 (Biotium 20070; to identify cell membrane and cytosol) by resuspending the cells in PBS and stain solution (1 uL/mL CellBrite NIR) in a 15 ml conical tube.

The cells were incubated with the stain for 30-60 minutes at 37° C. The cells were then pelleted by centrifugation at 300×g for 5 minutes and the stain mixture was removed to produce a pellet of stained cells for use in Example 1, above.

### Example 3—Cell Viability

Due to the synthetic nature of PEG components, PEG-based hydrogels are advantageous due to their relatively low lot-to-lot variability. However, hydrogels based entirely on PEG components are acidic, which can detrimentally impact the viability of 3D cell cultures in such hydrogels. Furthermore, adjusting the pH of PEG-only hydrogels into the neutral range can reduce their stiffness and density to the point where such hydrogels can no longer retain cells within them. Some specific gel formulations described herein utilize a 3.4k Da PEG-diacrylate, which retains the consistency (e.g., minimal lot-to-lot variability) of synthetic components as mentioned above. The pore size produced by 3.4 kDa PEGDA is large enough to allow diffusion of nutrients and dyes, but small enough to retain cells, including small immune cells which can be necessary for immunotherapy screening.

The viability of a tumor cell line (HCT-116) was evaluated at 3 and 5 days of growth in a Gelin-S and Ac-PEG-Ac-based gel.

Gelin gel was prepared as follows: 2.5% w/v 3.4 kDa Ac-PEG-Ac, 0.1% w/v Gelin-S, 0.25 mg/mL Laminin, and 0.1% w/v Irgacure 2959.

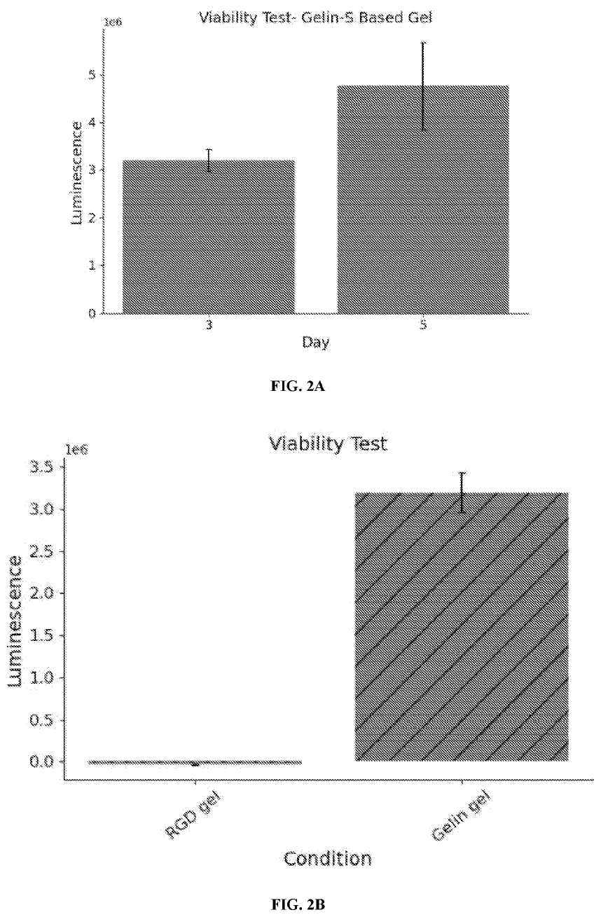

HCT-116 cells were resuspended in a gel at a concentration of 5M cells/mL. 5 μL of gel+cell mixture was added per well to 48 wells of two 384-well microplates (24 wells per plate). Gel was crosslinked by exposure to UV light for 15 s. 50 μL of Dulbecco's Modified Essential Media (DMEM), supplemented with 10% FBS, was added to each well. The microplates were then placed in a 5% CO₂ incubator at 37° C. 3 days after cell seeding, 1 microplate was removed from the incubator and a luminescence-based viability assay (CellTiter Glo 3D, Promega) was performed according to manufacturer specifications. Luminescence was read using a Varioskan plate reader (Thermo Scientific). Luminescence values were blank-subtracted, and average luminescence and standard error of the mean were calculated across all 24 samples. Mean luminescence after 3 days was 3,198, 142.9 with an S.E.M. of 229,246.8. After 5 days in culture, the second microplate was removed from the incubator and assessed for viability using the same luminescence assay. After 5 days, the mean luminescence was 4,754,192.3 with an S.E.M. of 913,546.9. This represents an increase of 48.7% over the values observed on day 3. FIG. 2A shows the growth of the HCT-116 cells in Gelin-S and Ac-PEG-Ac-based cells. These data suggest that cells in Gelin-S and Ac-PEG-Ac-based gels are viable at 3 days in culture and continue to grow through 5 days in culture.

### Example 4—Cell Viability: Comparison to PEG+RGD Hydrogels

Gelin-S+Ac-PEG-Ac (“Gelin”) hydrogels were compared to Ac-PEG-Ac+GRGDSPC (“RGD”) hydrogels.

Gelin gel was prepared as follows: 2.5% w/v 3.4 kDa Ac-PEG-Ac, 0.1% w/v Gelin-S, 0.25 mg/mL Laminin, and 0.1% w/v Irgacure 2959.

RGD gel was prepared as follows: 10% w/v 10 kDa Ac-PEG-Ac, 0.05% w/v Irgacure 2959, 2 mM GRGDSPC Peptide, and 0.25 mg/mL Laminin.

HCT-116 cells were resuspended in each gel type at a concentration of 5M cells/mL. For each gel type, 5 μL of gel+cell mixture was added per well to 24 wells of a 384-well microplate. Gel was crosslinked by exposure to UV light for 15 s. 50 μL of Dulbecco's Modified Essential Media (DMEM), supplemented with 10% FBS, was added to each well. Microplates were then placed in a 5% C02 incubator at 37 C. 72 h after cell seeding, microplates were removed from the incubator and a luminescence-based viability assay (CellTiter Glo 3D, Promega) was performed according to manufacturer specifications. Luminescence was read using a Varioskan plate reader (Thermo Scientific). Luminescence values were blank-subtracted, and average luminescence and standard error of the mean were calculated across all 24 samples for each gel type. Average luminescence for RGD-based gel samples was −3,1647.3 with an S.E.M. of 4,128.5, while average values for Gelin samples was 3,198, 142.9 with an S.E.M. of 229,246.8. FIG. 2B shows a comparison between Gelin-S-based gel and RGD-based gel. These data suggest that cells in the RGD condition were not viable after 3 days in culture, while cells in the Gelin condition remained viable.

### Example 5: Cell Viability: Comparison to PEG+RGD Hydrogels and PH Balanced PEG+RGD Hydrogels

As observed in Example 4, cells do not survive in RGD gels. It was observed that the typical pH of RGD gels was in the 4-5 range, and hypothesized that the low pH may be negatively impacting cell viability. To test whether RGD gels could be improved by adjusting the pH into the physiological range of 7.4, 1M NaOH was added dropwise to RGD gel formula (above) and measured the pH after each addition. The total amount of NaOH added was negligible relative to the total volume of the gel. These pH balanced RGD-based hydrogels (“Balanced RGD”) were compared to Gelin-S+Ac-PEG-Ac (“Gelin”) hydrogels and Ac-PEG-Ac+GRGDSPC (“RGD”) hydrogels without pH balancing. The Gelin and RGD-based gels were prepared as above. The pH of Gelin gels was measured at 7.3 without any modifications. HCT-116 cells were resuspended gel at a concentration of 5M cells/mL. 5 μL of gel+cell mixture was added per well to 72 wells of 3 384-well microplates (24 wells per plate). Gel was crosslinked by exposure to UV light for 15 s. 50 μL of Dulbecco's Modified Essential Media (DMEM), supplemented with 10% FBS, was added to each well. The microplates were then placed in a 5% C02 incubator at 37 C. 3 days after cell seeding, the microplates were removed from the incubator and a luminescence-based viability assay (CellTiter Glo 3D, Promega) was performed according to manufacturer specifications. Luminescence was read using a Varioskan plate reader (Thermo Scientific). Luminescence values were blank-subtracted, and average luminescence and standard error of the mean were calculated across all 24 samples. Mean luminescence after 3 days was −31647.26 with an S.E.M. of 4128.54 for the RGD condition; 3206783.46 with an S.E.M. of 232717.981 for the Balanced RGD condition, and 3198142.86 with an S.E.M. of 229246.80 for the Gelin condition. These data suggest that a neutral pH in the physiological range is important to maintain cell viability. The PH balanced RGD gels and the Gelin gels both meet this requirement. However, unlike the Gelin gels, it was observed that RGD gels with a neutral pH did not maintain their structure after several days in culture. FIG. 2C shows a viability comparison between Gelin-S-based gel, RGD-based gel, and PH balanced RGD gel. By day 5, the pH balanced RGD gels had dissolved completely, allowing the cells to fall through the gel and adhere to the plate bottom. In addition, pH balancing these gels requires an additional step and can be difficult to automate. This demonstrates that Gelin gels have the desired combination of lasting structural integrity and neutral pH.

### Example 6: Hydrogel Plating

Due to the small size of the well and the polar nature of the hydrogel liquids in question, the hydrogel liquids tend to stick or wick onto one or two corners of the well rather than even cover the well surface. Many of the steps undertaken, including the double layer, are designed to avoid this scenario because it causes major problems when collecting and analyzing image-based data. Gel components for the first layer were mixed together and an equal volume was added to all 8 wells in column 1 of a standard U-bottom 96 well microplate. An Opentrons liquid handling robot, running on a custom program and configured with a 2nd-generation P20 8-channel pipette, was used to seed a flat-bottom 384-well microplate as follows: first, the robot simultaneously aspirated 20 microliters (uL) of gel from the 96-well plate, and moved to the 384-well plate so that the tips were positioned 0.2 mm above the bottom of wells A1, C1, E1, G1, I1, K1, M1, and O1. Each channel then dispensed 20 μL in order to evenly coat the well bottom with gel, waited 0.5 seconds, and then aspirated 15 μL, leaving a 5 μL layer of gel behind. The robot then returned to column 1 of the 96-well source plate, aspirated 5 μL (so that the tip now contained 20 μL), and moved to wells B1, D1, F1, H1, J1, L1, N1, and P1 of the 384-well plate. The robot then dispensed 20 μL and removed 15 μL as before, so that now all wells in column 1 of the 384-well plate now contained a 5 μL layer of gel. This process was repeated for the remaining 23 columns of the 384-well plate. Total time for this procedure was roughly 20 minutes. The plate was then exposed to UV light for 15 seconds to crosslink the gel. Next, components for the second layer of gel were mixed together along with cells from a patient sample and added in equal volumes to each well in column 2 of the 96-well source plate. The seeding process as written above was then repeated for the second layer of gel, such that at the end of the process each well in the 384-well plate was given a second 5 μL layer of gel containing patient cells. Again, this process took roughly 20 minutes. The plate was then exposed to 10 seconds of UV light. From start to finish, the entire procedure lasted roughly 1 hour.

### Example 7: Hydrogel Plating

A 384 well microplate was placed on to the plate carrier of a MultiFlo FX automated liquid dispenser equipped with a 5 μL, 8-channel peristaltic pump cassette. A 50 milliliter (mL) conical tube filled with 40 mL of deionized water was mounted near the dispenser and the end of the cassette tubing was placed into the water and primed to fill the tubing dead volume with the water to be dispensed. Using a custom program, 15 microliters (μL) of water was dispensed into each well of the 384-well plate. The dispense process lasted roughly 30 seconds. Water was then purged from the tubing. Next, the liquid in the wells was manually flicked out, leaving behind only a residue of water coating the well bottom. Then, gel components for the first layer of hydrogel were mixed together in a 50 mL conical tube. The tube was then mounted near the MultiFlo the end of the cassette tubing placed into the gel and primed. Using a custom program, 5 μL of gel was dispensed into each well of the 384-well plate, a process which took roughly 15 seconds. The plate was then crosslinked under a UV light for 15 seconds. Next, components for the second layer of gel were mixed together along with cells from a patient sample and added to a second 50 mL conical tube. As with the first layer, 5 μL was dispensed into each well of the microwell plate. Afterwards, the gel was crosslinked with 10 seconds of exposure to UV light. This process lasted roughly 20 minutes from start to finish.

### Example 8: Hydrogel and Hydrogel Plating without Laminin

A hydrogel was created substantially as described in Example 1, except that the following components were used as set forth in Table 2.

| | AC-PEG-AC, 3.4 kDa, 10% | |
|---|---|---|
| | w/w dissolved in an aqueous | Gelin-S, 2% w/w dissolved |
| | solution of 0.1% w/w | in an aqueous solution of |
| Component | irgacure in PBS | 0.1% w/w irgacure in PBS |
| Vol. per mL | 0.5 | 0.5 |

100 mg of the AC-PEG-AC (e.g., Formula I) was added to a 1.5 mL microcentrifuge tube. A 0.1% irgacure solution was added to the tube containing AC-PEG-AC and mixed. A 50 mg bottle of Gelin-S was resuspended in 2.5 mL of a 0.1% irgacure solution. The AC-PEG-AC solution and the Gelin-S mixture were combined and immediately plated into a 384-well source plate. Immediately after the first layer was plated, it was cured with UV light (320 nm-390 nm) for 15 seconds with an intensity of 250 mW/cm².

Cells were stained as described in Example 2 and centrifuged at 300×g for 5 minutes to create a cell pellet. The supernatant was removed from the cell pellet.

To form a second cured hydrogel layer, the AC-PEG-AC solution and the Gelin-S mixture were combined as set forth in Table 2 with the pellet of stained cells. The stained cells were re-suspended in the gel mixture to create a seeded gel mixture. The seeded gel mixture was plated on top of the first layer and immediately cured with UV light (320 nm-390 nm) for 15 seconds with an intensity of 250 mW/cm².

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for producing a three-dimensional hydrogel system in a well, the method comprising:
(a) delivering a first mixture comprising a first acrylate and a first thiolated polymer into a well, thereby forming a concave meniscus within the well;
(b) cross-linking the first acrylate and the first thiolated polymer of the first mixture to form a first cured hydrogel layer, wherein the first cured hydrogel layer comprises a concave upper surface;
(c) delivering a second mixture comprising a second acrylate and second thiolated polymer into the well on top of the first cured hydrogel layer;
(d) cross-linking the second acrylate and the second thiolated polymer of the second mixture to form a second cured hydrogel layer on top of the first cured hydrogel layer, wherein the second cured hydrogel layer comprises an upper surface that is less concave than the concave upper surface of the first cured hydrogel layer.

2. The method of claim 1, wherein the upper surface of the second cured layer is substantially planar.

3. The method of claim 1 or claim 2, wherein crosslinking the first acrylate and the first thiolated polymer to form the first cured hydrogel layer comprises exposing the first mixture to ultraviolet light while the first mixture is disposed within the well and before the second mixture has been delivered to the well.

4. The method of any preceding claim, wherein crosslinking the second acrylate and the second thiolated polymer to form the second cured hydrogel layer comprises exposing the second mixture to ultraviolet light while both the first cured hydrogel layer and the second mixture are disposed within the well.

5. The method of claim 4, wherein the first mixture is exposed to ultraviolet light for a period of time that is longer than a period of time to which the second mixture is exposed to ultraviolet light.

6. The method of claim 5, wherein the second mixture is exposed to ultraviolet light for less than 12 seconds and the first mixture is exposed to ultraviolet light for greater than 12 seconds.

7. The method of any preceding claim, wherein the first mixture and the second mixture are exposed to ultraviolet light from 10 seconds to 20 seconds.

8. The method of any one of claims 4-7, wherein the ultraviolet light has an average wavelength from 320 nm and 390 nm.

9. The method of any one of claims 4-8, wherein the ultraviolet light has an intensity of between 200 and 300 mW/cm².

10. The method of any preceding claim, wherein the second mixture further comprises a plurality of cells.

11. The method of any preceding claim, wherein the plurality of cells are unaggregated cells.

12. The method any one of claims 10-11, wherein the plurality of cells have been stained prior to (c).

13. The method of claim 12, wherein the plurality of cells that have been stained have been stained with one or more of (i) a dye specific to cell nuclei, (ii) a dye specific to cell membranes, (iii) a dye specific to live cells, and (iv) a dye specific to dead cells.

14. The method of claim 13, wherein the plurality of cells that have been stained have been stained with one or more of a lipophilic carbocyanine dye, a biz-benzimide, a xanthene dye, or propidium iodide.

15. The method of claim 10 or claim 11, wherein the plurality of cells have been marked with a fluorescent tag prior to (c).

16. The method of any preceding claim, wherein the second mixture does not include laminin.

17. The method of any one of claims 1-15, wherein the second mixture further comprises laminin such that laminin is non-covalently distributed within the second cured hydrogel.

18. The method of any preceding claim, wherein the first mixture further comprises a photoinitiator.

19. The method of claim 18, wherein the photoinitiator comprises 2-Hydroxy-4′-(2-hydroxyethoxy)-2-methylpropiophenone.

20. The method of any preceding claim, wherein the second mixture further comprises a photoinitiator.

21. The method of claim 20, wherein the photoinitiator comprises 2-Hydroxy-4′-(2-hydroxyethoxy)-2-methylpropiophenone.

22. The method of any preceding claim, further comprising adding cell culture media to the three-dimensional hydrogel system.

23. The method of claim 22, wherein the cell culture media comprises one or more of sodium phosphate, potassium chloride, sodium bicarbonate, ferric nitrate, calcium chloride, or potassium chloride.

24. The method of any preceding claim, wherein the kinematic viscosity of the first mixture is, when delivered to the well, between 0.5 mm²/s to 10 mm²/s.

25. The method of any preceding claim, wherein the kinematic viscosity of the second mixture is, when delivered to the well, between 0.5 mm²/s to 10 mm²/s.

26. The method of any preceding claim, wherein one or both of the first acrylate and the second acrylate is a multi-armed PEG acrylate.

27. The method of any preceding claim, wherein one or both of the first acrylate and the second acrylate is PEG diacrylate.

28. The method of claim 27, wherein the PEG diacrylate of the first mixture has a number average molecular weight of greater than three kilodaltons.

29. The method of claim 28, wherein the PEG diacrylate of the first mixture has a number average molecular weight of between three and four kilodaltons.

30. The method of claim 27, wherein the PEG diacrylate of the second mixture has a number average molecular weight of greater than three kilodaltons.

31. The method of claim 30, wherein the PEG diacrylate of the second mixture has a number average molecular weight of between three and four kilodaltons.

32. The method of any preceding claim, wherein one or both of the first thiolated polymer and the second thiolated polymer is thiolated gelatin.

33. The method of claim 32, wherein the thiolated gelatin has a degree of thiolation between 20% and 30%.

34. The method of any one of claims 27-33, wherein the weight ratio of PEG diacrylate to thiolated gelatin in the second mixture is between 1:4 and 2:1, such as 1:2.

35. The method of any one of claims 27-33, wherein the weight ratio of PEG diacrylate to thiolated gelatin in the second mixture is between 1:1 and 8:1, such as between 2:1 and 8:1.

36. The method of any one of claims 27-35, wherein the second mixture comprises 2-Hydroxy-4′-(2-hydroxyethoxy)-2-methylpropiophenone at a weight ratio of 2-Hydroxy-4′-(2-hydroxyethoxy)-2-methylpropiophenone to thiolated gelatin of between 1:20 and 1:100, such as 1:50.

37. The method of any one of claims 27-36, wherein the second mixture comprises laminin at a weight ratio of thiolated gelatin to laminin of between 1:1 and 8:1, such as 4:1.

38. The method of any one of claims 27-37, wherein the concentration of laminin in the second mixture is at least 0.1 mg/mL.

39. The method of any one of claims 27-38, wherein the concentration of laminin in the second mixture is no more than 1.0 mg/mL.

40. The method of any one of claims 38-39, wherein the concentration of laminin in the second mixture is between 0.2 and 1.0 mg/mL or between 0.2 and 0.6 mg/mL.

41. The method of any preceding claim, wherein the well is one well of an array of wells on a multi-well plate.

42. The method of claim 41, wherein the array of wells comprises at least 96 wells.

43. The method of claim 41, wherein the array of wells comprises at least 384 wells.

44. The method of claim 41, wherein the array of wells comprises at least 1536 wells.

45. The method of claim 41, wherein steps (a) through (d) are completed over 384 wells in less than one hour.

46. The method of claim 41, wherein steps (a) through (b) are completed over 384 wells in less than 20 minutes.

47. The method of claim 41, wherein steps (c) through (d) are completed over 384 wells in less than 20 minutes.

48. The method of any one of claims 1-47, wherein delivering the first mixture comprises delivery via a robotics system.

49. The method of any one of claims 1-48, wherein delivering the first mixture comprises (i) dispensing a first amount of the first mixture to the well and (ii) aspirating a second amount of the first mixture from the well.

50. The method of claim 49, wherein the first amount is greater than the second amount.

51. The method of any preceding claim, wherein (b) comprises exposing the well to UV light.

52. The method of any preceding claim, wherein delivering the second mixture comprises delivery via a robotics system.

53. The method of any preceding claim, wherein delivering the second mixture comprises (i) dispensing a first amount of the second mixture to the well and (ii) aspirating a second amount of the second mixture from the well.

54. The method of claim 53, wherein the first amount is greater than the second amount.

55. The method of any preceding claim, wherein (d) comprises exposing the well to UV light.

56. The method of any one of claims 1-55, wherein the second mixture comprises cells.

57. The method of claim 56, wherein the cells are substantially evenly distributed throughout the second cured hydrogel layer.

58. A hydrogel formed by the method of any one of the above claims.
